(19) United States
(12) Patent Application Publication
(10) Pub. No.: US 2010/0047801 A1
(43) Pub. Date: Feb. 25, 2010

(54) METHOD AND SYSTEM FOR DATA DRIVEN MANAGEMENT OF INDIVIDUAL SEEDS

(75) Inventors: JASON COPE, Ankeny, IA (US); JOHN C. HOEKSTRA, Johnston, IA (US); JAMES DAVID DIMOND, Ames, IA (US); STEVEN M. BECKER, Johnston, IA (US); DAVID E. BOWEN, Grimes, IA (US); TIMOTHY P. MEYER, Des Moines, IA (US); JAMES L. HUNTER, Ankeny, IA (US); BARRY L. STOTT, Winterset, IA (US); LOREN STEENHOEK, Ankeny, IA (US); JASON J. KELSICK, Alleman, IA (US); DAVID C. SMITH, Ankeny, IA (US); JOSHUA L. MONGAN, ST. CHARLES, IA (US)

Correspondence Address: MCKEE, VOORHEES & SEASE, P.L.C.;ATTN: PIONEER HI-BRED, 801 GRAND AVENUE, SUITE 3200, DES MOINES, IA, 50309-2721, US

(73) Assignee: PIONEER HI-BRED INTERNATIONAL, INC., Johnston, IA (US)

(21) Appl. No.: 12/533,145

(22) Filed: Jul. 31, 2009

Related U.S. Application Data
(60) Provisional application No. 61/090,961, filed on Aug. 22, 2008.

(51) Int. Cl. C12Q 1/68; G06F 17/30

(52) U.S. Cl. 435/6; 707/104.1; 707/E17.044

(57) ABSTRACT

A method for managing seed includes non-destructively sampling individual seeds to assist in providing evaluation data for each of the individual seeds, storing the evaluation data and seed identifiers associated with each of the individual seeds in a data store, selecting a subset of seed for planting at least partially based on the evaluation data, and planting the subset of seed. A system for management of seed on an individual seed basis includes an evaluation subsystem for evaluating individual non-destructively sampled seeds to provide evaluation data for the seeds, a selection subsystem for selecting a subset of the individual seeds at least partially based on the evaluation data, and a planting subsystem for planting the subset of individual seeds.

### RELATED APPLICATIONS

This application claims priority under 35 U.S.C. §119 to U.S. Patent Application No. 61/090,961, filed Aug. 22, 2008, hereby incorporated by reference in its entirety.

### FIELD OF THE INVENTION

The present invention relates to managing operations associated with plant breeding, and more particularly to managing operations associated with seeds on an individual seed basis.

### BACKGROUND

A primary goal of seed companies is to develop seed that grow into plants that are commercially desirable to crop producers. Seed companies devote substantial resources towards research and development of commercially desirable seed.

Conventional research and development techniques tend to be laborious and require vast amounts of land and space. All or much of the seed involved in the research is planted in research plots. After plants emerge from the seed, tissue samples from each plant are acquired. The tissue samples are transported to a laboratory to determine information needed for the research and development of the seed and plants from the seed. These methods are well-known in the industry. The resource costs of land, labor, and machinery are substantial.

Therefore, a need exists in the industry to materially reduce the resources used for evaluating plants and their seed for potential commercial production or further use in plant and seed research and development.

### SUMMARY

A method for managing seed includes non-destructively sampling individual seeds to acquire evaluation data for each of the individual seeds, storing the evaluation data and seed identifiers associated with each of the individual seeds in a data store, selecting a subset of the sampled seeds for planting at least partially based on the evaluation data, and planting the subset of seeds.

A system for management of seed on an individual seed basis includes an evaluation subsystem for evaluating individual non-destructively sampled seeds to provide evaluation data for the seeds, a selection subsystem for selecting a subset of the individual seeds at least partially based on the evaluation data, a planting subsystem for planting the subset of individual seeds, and a data store for storing seed management data, the seed management data including the evaluation data.

### BRIEF DESCRIPTION OF THE DRAWINGS

FIG. 1 illustrates an overview of a method for managing seed.

FIG. 2 illustrates a system for managing seed.

FIG. 3 illustrates seed movement throughout a process.

FIG. 4A and FIG. 4B illustrate data associated with different steps in a process.

FIG. 5 illustrates the method in the context of corn.

FIG. 6 provides a detailed example of the non-destructive evaluation and seed selection steps.

FIG. 7A to FIG. 7C illustrate one manner in which seed may be stored.

### DETAILED DESCRIPTION

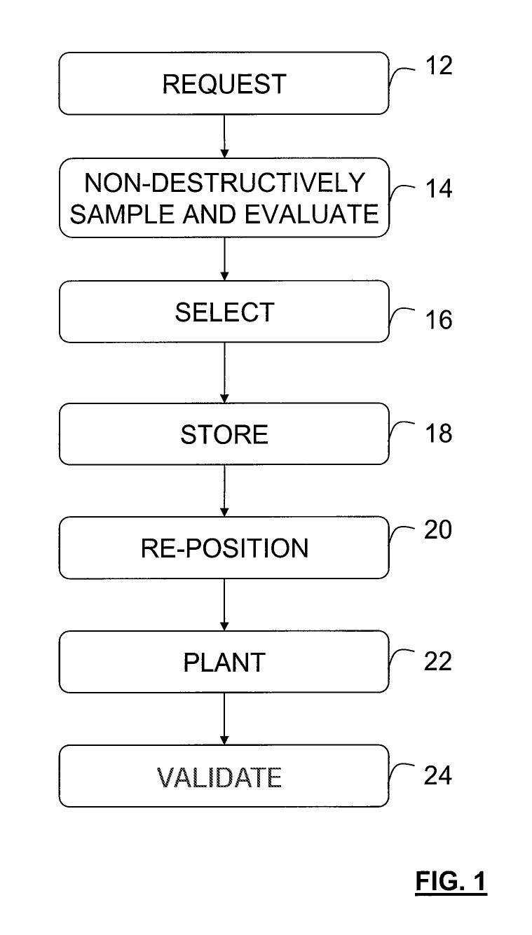

FIG. 1 illustrates an overview of a method for managing seed. In FIG. 1, in step 12 a request is formulated. The request identifies one or more seeds of interest for testing. In step 14, non-destructive sampling and evaluation of seed occurs. The non-destructive sampling provides for seed that remains suitable for planting and development into a plant after the sampling of the seed. Thus, non-destructive sampling allows seeds which have tissue removed to still be planted and grown to a plant. In other words, samples are taken from a seed and the seed remains germination viable. The removed tissue need not be preserved and can, in fact, be destroyed in whole or in part in different tests or assays. It is to be further understood that not every single seed need be sampled. Instead one of a group of seeds may be sampled and seeds from the group may be planted, especially when an individual seed is representative of a genetically uniform population.

The seed samples of the individual seeds are evaluated to provide genetic information such as phenotype and/or genotype data. Different types of data may be obtained. Of particular interest may be the identification of genetic markers associated with desirable traits. Examples of such traits may include yield traits, disease resistance traits, insect resistance traits, herbicide resistance traits, or tolerance to environmental stresses. In the process of evaluating the seed, any number of forms of analysis may be performed on the tissue sample. The analysis may be genetic, chemical or physical in nature. In addition, the evaluation data may be combined with other data in order to make seed selection decisions.

Where the seed sample is evaluated for genetic data, examples of types of genetic evaluation may include, without limitation, determining if the seed includes a particular genetic marker, determining if the seed includes a particular seed nucleotide polymorphism, determining if the seed includes a particular restriction fragment length polymorphism, a particular haplotype, a particular tag SNP, alleles of a particular genetic marker, a particular gene, a particular DNA-derived sequence, a particular RNA-derived sequence, a particular promoter, a particular siRNA, a particular QTL, a particular transgene or otherwise performing a genetic evaluation.

In step 16, selection occurs. Selection is an important aspect of the plant breeding process. The selection is at least partially based on evaluation data developed during the step of evaluating the seed tissue. Additional data may also be relied upon such as parent information, parent genotype, or other information of potential relevance to plant characteristics of interest. Note that selection occurs prior to the seed being planted. This provides a number of advantages over a process that requires planting and growing of seeds in order to evaluate the seeds, traits, or other characteristics. Efficiencies are achieved because seeds can be excluded without planting them. Thus, fewer resources need be devoted to less promising candidates and more resources can be devoted to more promising candidates. This is an important advantage as growing out seed can use significant resources.

In step 18, the selected individual seeds are stored. The individual seeds may be stored in an indexed system so that the location of any individual seed is known. Thus, the storage system used typically isolates each individual seed from other individual seeds, so that the identity of each individual seed may be maintained. Knowing the identity of each individual seed allows one to store and access data about each individual seed throughout the entire process.

In step 20, the individual seeds may be repositioned. Repositioning of the individual seeds is an optional, but sometimes desirable step. It is contemplated that after seed are stored, it may be desirable to re-index and/or reposition the seed before planting. Doing so may be desirable for several reasons. First, the individual seeds may be re-arrayed into a container which may be directly used by a planter. Second, it is desirable to place the seeds in a particular configuration for planting to gain additional efficiencies. The configuration may include relative positions of the seeds, thus a first seed may be placed next to a second seed where doing so may make the cross pollination process more efficient, or where isolation is necessary.

In step 22, planting of the seeds occurs. Planting of the seed may be performed with an automated single seed planter which is adapted to plant the seeds according to a pre-defined configuration. In addition the automated single seed planter is preferably adapted to collect as-planted data during planting. The as-planted data may include time and position information, such as may be provided by a global positioning system (GPS) receiver. In addition, the as-planted data may include data associated with planting operations. It is further contemplated that exceptions to the pre-defined configuration may occur. Thus, the planter may be further adapted to identify exception conditions which occur during planting.

In step 24, a validation operation is performed. The validation operation may involve a comparison to a pre-determined arrangement for the seeds to as-planted data collected from the planter. It is to be understood, that there may be exceptions to the pre-determined arrangement that occur for any number of reasons. Thus, the validation step allows for the configuration of the seed to be updated based on the as-planted data if necessary.

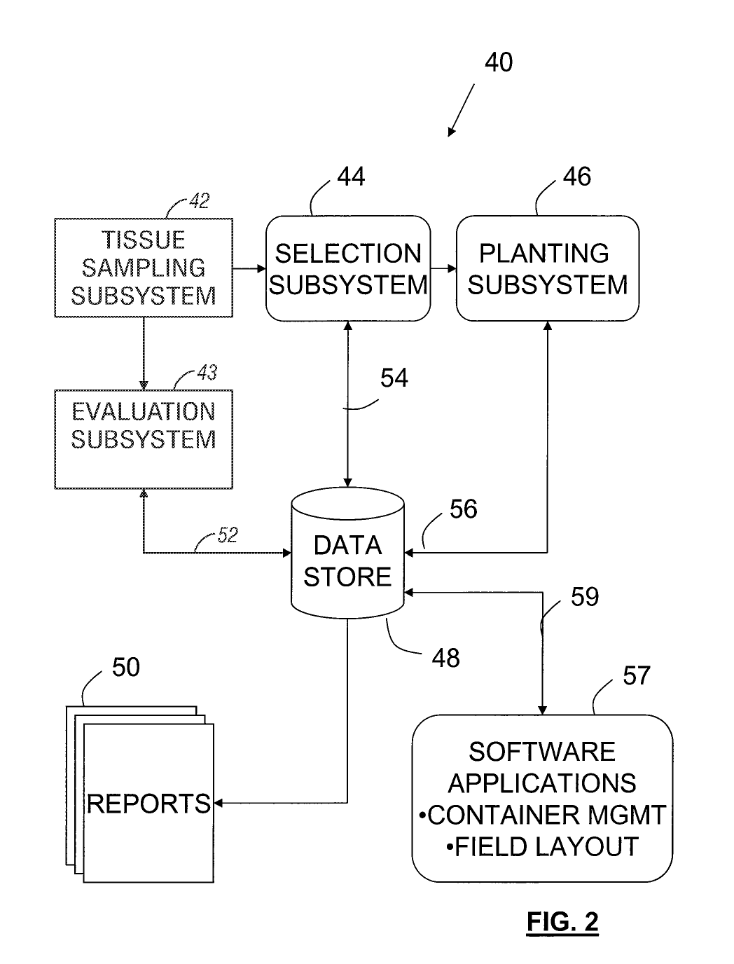

FIG. 2 illustrates a system 40. In FIG. 2, a tissue sampling subsystem 42 and an evaluation subsystem 43 are shown. These subsystems are in operative communication with a data store 48. The data store 48 may be a logical data storage and may be located at or across any number of locations. A selection subsystem 44 is also in operative communication with the data store 48. A planting subsystem 46 is also in operative communication with the data store 48. The subsystems may also be in operative communication to each other. For example, there may be a data path 52 between the subsystem 42 and the data store 48. There may be a data path 54 between the selection subsystem 44 and the data store 48. There may be a data path 56 between the planting subsystem 46 and the data store 48. In the system 40, data collected by the different subsystems as well as data which are used by the different subsystems are stored in the data store 48. The data within the data store 48 may be used for a number of different purposes. The data within the data store 48 may be used to drive the process by making available to each subsystem data associated with each individual seed. In addition, the data within the data store 48 may be used for other purposes, including use in various types of reports 50 which may be made available to uses. The reports 50 may provide for outputting data regarding status of individual seeds, outputting data to assist in regulatory reporting, and outputting data for use in data analysis. In addition, as shown in FIG. 2, software applications 57 may access the data store 48 such as through the data path 59. One example of such an application is a container management application which may be used to track the location of containers. Examples of containers may include seed storage trays, planter trays, sample containers, and other types of containers. Another example of such an application is a field layout tool which may be used to determine where to plant particular types of seeds within one or more plots or fields. Of course, any number of other types of software applications may have access to the data store 48.

It should be understood that a data store 48 need not be a physical location, but may be a logical location. A data store may include a logical grouping of data without regard of where it is stored, and which may be stored across multiple locations. Examples of such locations may include, without limitation, one or more database servers, PLC memories, RFID tags, reports, or other locations where data is stored, or any combinations of such locations

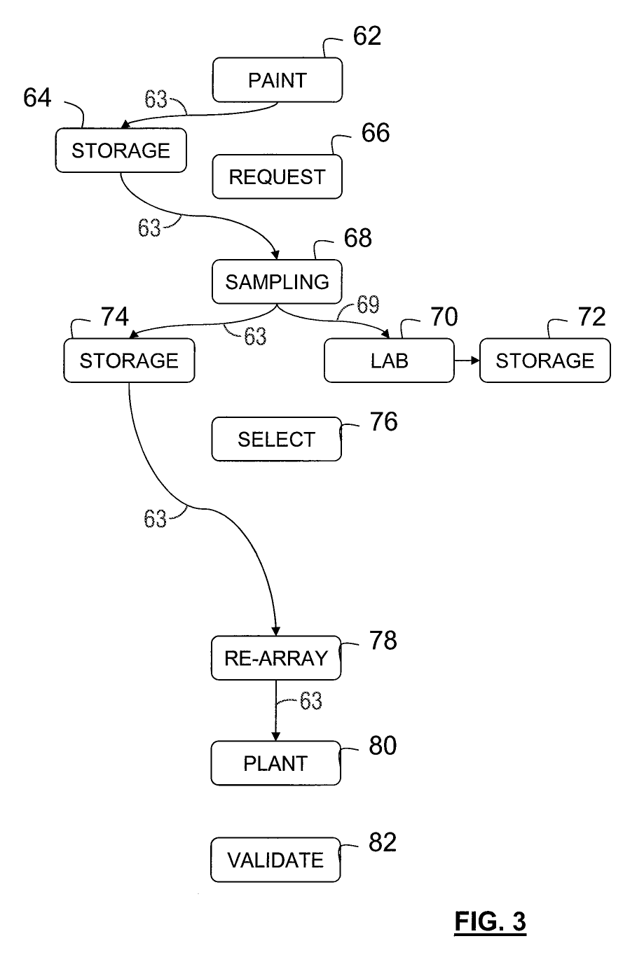

FIG. 3 illustrates seed movement throughout a process. In step 62, a paint step occurs. The paint step is an optional step in which a magnetic paint may be applied to a seed 63. The magnetic paint assists in orienting the seed for automated seed handling processes. Of course, other methods of orienting the seeds may be used, including manual or mechanical means. After the paint step 62, the seed may be placed in storage 64 until a request is made in step 66 is made for testing the seed. Once the request is made in step 66, the seed 63 is conveyed from the storage 64 for sampling in step 68. The sampling step 68 provides for procuring a sample 69 of the seed 63 without compromising the seed's suitability for planting and normal development. The seed sample 69 is conveyed to a laboratory 70 for analysis. If not all of the sample 69 is used, the remaining portion of the sample 69 may be stored in storage 72 or sent to another laboratory for further testing. After the sampling, the seed itself is conveyed to storage 74. At some point a selection step 76 occurs. Selected seed is re-arrayed from the storage 74 during a re-arraying step 78. The selected seed may then be conveyed to a planter for the planting step 80 or to a greenhouse for planting. A validation step 82 may then occur to validate using the data acquired during the planting step 80.

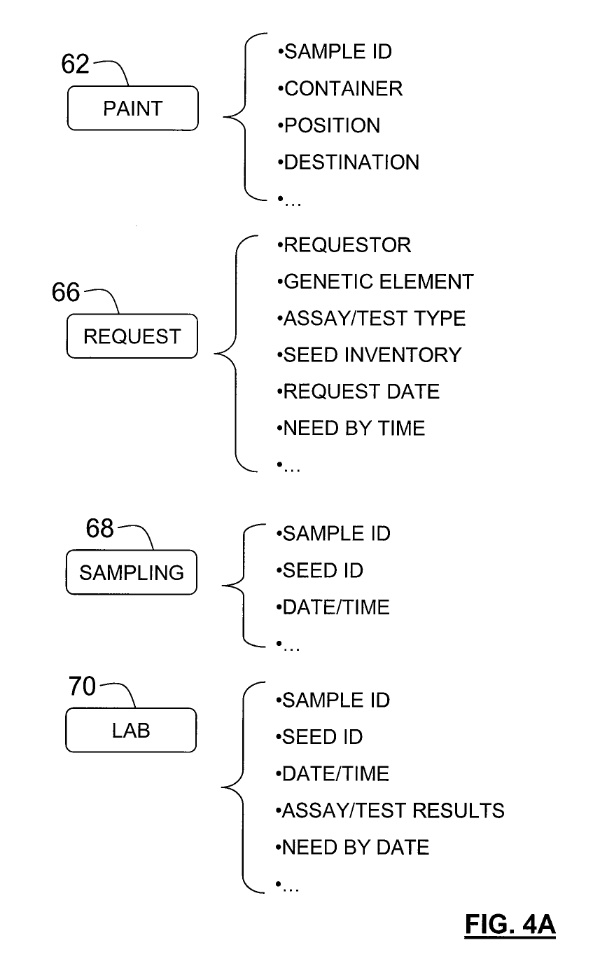

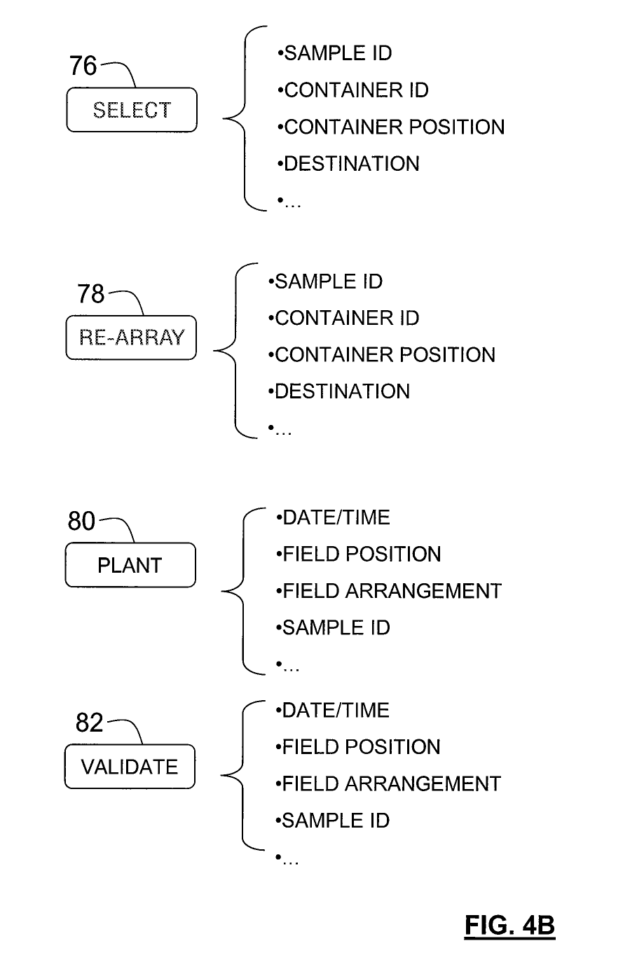

FIGS. 4A and 4B illustrate data associated with different steps. In FIG. 4A, a paint step 62 is shown. Data which may be collected at or associated with the paint step includes, without limitation, a sample ID, a container ID, a position, and a destination for the seeds.

A request step is also shown. Data which may be collected at or associated with the request step includes, without limitation, a requestor, a genetic element, an assay or test type, a seed inventory, a request date indicative of the date and/or time a request is made, and a need by time to indicate when the test results are needed.

A sample step 68 is also shown. Data which may be collected at or associated with the sample step 68 may include, without limitation, a sample ID, a seed ID, and a date/time that a sample is taken, assay or test type requested, sample location, and/or seed location. Note that where every seed is individually sampled, the sample ID and the seed ID could be the same.

A lab analysis step 70 is also shown. Data which may be collected at or associated with the lab analysis step 70 may include, without limitation, a sample ID, a seed ID, a date/time of the analysis, results of any assays or tests, and a need by date.

A selection step 76 is shown in FIG. 4B. Data which may be determined at or associated with the selection step 76 may include, without limitation, a sample ID, a container ID, a seed location and/or container position to identify the position of seed within a container identified with the container ID. In addition a destination (physical and/or logical) may be determined or collected at the selection step 76.

A re-array or re-positioning step 78 is shown in FIG. 4B. Data which may be collected at or associated with the re-array step 78 may include, without limitation, a sample ID, a container ID, a container position to identify the position of a seed within the container identified with the container ID. In addition a destination (physical and/or logical) may be determined or collected at the re-array step 78.

A planting step 80 is also shown in FIG. 4B. Data which may be collected at or associated with the planting step 80 may include, without limitation, date/time data associated with planting operations, field experiment ID, field experiment protocol(s), field location, field characteristic(s) field position for each seed (individually or by type), and/or field arrangement which may include relative positions of seeds within a field and may include geospatial position of seeds within the field. In addition, a sample ID may be associated with the data for each sample.

A validate step 82 is also shown in FIG. 4B. Data which may be collected at or associated with the validate step 82 may include, without limitation: date/time data, field position data, field arrangement data, and sample ID data.

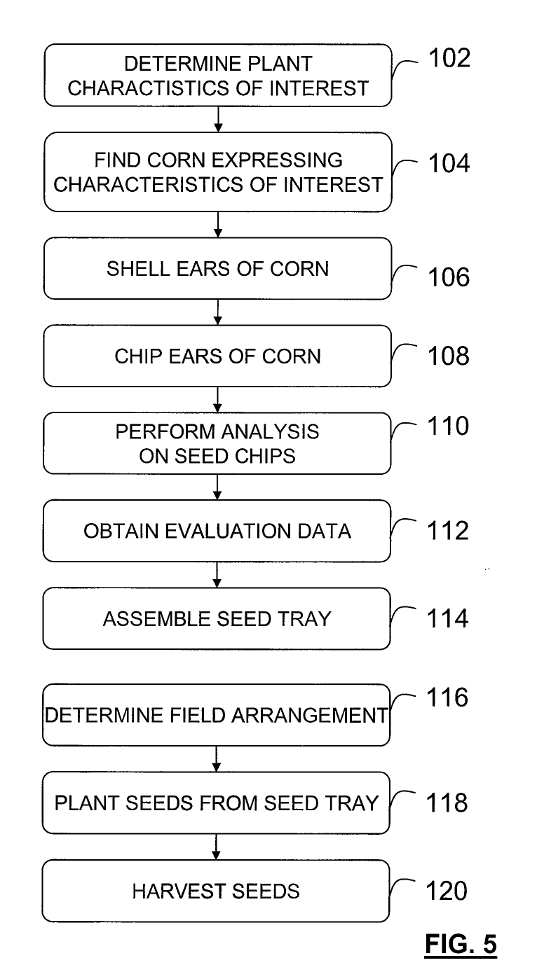

FIG. 5 illustrates another example of a method in the context of corn, which is one of the types of seeds which may be used. It is to be understood, however, that this example is only intended to illustrate one application of the invention. The invention can be utilized for other seed and other objects. The range of sizes can vary as well as the nature of the object. As will be understood by one of skill in the art, the embodiments of the invention will be used with seed that are of convenient size to be sampled. Some seed are extremely fine and small, somewhat like dust particles or grains of salt, while others are particularly large and hard, such as the seed from the Lodoicea maldivica palm, which are 20 to 24 pounds in weight. One of skill in the art recognizes that seed intended to be used with the embodiments of the invention must be of a size and weight that allow convenient sampling. Such seed include, but are not limited to, many agriculturally important seed such as seed from maize (corn), soybean, Brassica species, canola, cereals such as wheat, oats or other grains, and various types of vegetable and ornamental seed. Analogous applications will be obvious from this example and variations obvious to those skilled in the art will be included.

Furthermore, the method is not strictly limited to seeds and would be useful for other propagative structures including meristemic tissue such as sprouted plants, tuber eyes, and the like.

In step 102, a determination is made as to plant characteristics of interest or potential interest. In step 104, corn seed having or potentially having the characteristics of interest is identified. In step 106, the ears of corn are shelled. In step 108, seed chipping (one type of seed sampling) is performed. In step 110, DNA or other types of analysis is performed on the seed chips. In step 112, genotype data is obtained, for example DNA sequences are identified, the presence or absence of particular genetic markers are confirmed and/or or other evaluation data or test results are obtained. In step 114, a seed tray is assembled which includes seeds which have been evaluated. In step 116, a field arrangement which includes seeds of interest is determined. In step 118, the seeds within the seed tray are planted. In step 120, the seeds are harvested. The harvest process in step 120 may use the as-planted data to assist in identifying position of the seed within the field.

FIG. 6 provides a detailed example of a method 200 for the non-destructive evaluation and seed selection steps of FIG. 1. In particular FIG. 6 shows one example of the non-destructive evaluation step 14 and the selection step 16. The method 200 allows selection of seeds to use for further research or commercial production without growing plants from the seeds and testing living tissue of the plants. The method can avoid use of land, labor, time, equipment, and materials for growing plants from the seeds to then acquire non-destructive samples to analyze for selection decisions. The method can be non-destructive of the seeds, it can allow relatively high throughput of multiple samples, and/or be substantially automated. The method 200 can include the following steps.

A plurality of seeds, for example, corn kernels of different genotypes are analyzed and compared for the purpose of identifying and selecting whether any will be utilized for further research and development or planted to produce commercial or research scale quantities. The method applies as well to other seed specific tests or analyses, as will be apparent to the skilled artisan.

In step 201 candidate seed is identified. One or more factors are used to decide which seed will be a candidate seed for evaluation. In this example, a set of individual candidate seeds, each having a different trait and/or genotype and/or corn variety, are pre-selected. Each candidate seed is isolated from the other candidates but associated with information from which the candidate seeds may be identified in step 202. The identity of each seed may be maintained through the method. Each seed may be identified with an identifier or other code which is stored in the data store 48 or other computer accessible storage. Other methods are possible. In step 203, each candidate seed is assigned an identifying label, such as an identification number.

Pre-selection of candidate seeds can be based on any of a number of factors or criteria. Research scientists may select the factors or criteria used. Examples of types of factors and criteria are commonly known in the art. Some such examples include genotype, phenotype, parentage, traits, or characteristics. Further discussion of these factors or criteria can be found in such references as: (a) Chahal, G. S & Gosal, S. S., 2002. “Principles and Procedures of Plant Breeding”, Alpha Science International, United Kingdom; (b) Falconer, D. S. 1989. “Introduction to Quantitative Genetics”. 3rd Ed. Longman. Burnt Mill; and (c) Frisch, M. & Melchinger, A. E., 2005. “Selection Theory for Marker-assisted Backcrossing.” Genetics 170:909-917; which are incorporated by reference herein.

In step 204 a single candidate seed is isolated by any of a number of ways to present it for removal of specific tissue to gain access to, expose, or sample certain specific tissues(s), part(s), or structure(s) of that seed for testing, or collect the removed tissue for testing. For purposes of this description, tissue(s), part(s), or structure(s) of a seed will collectively sometimes be referred to as tissue. One example of isolation is to place the candidate seed into a cavity or well. Another is to grasp, hold, or restrain the seed by or to some device (e.g. with a vacuum; by clamping action). Another is to apply a substance to the seed which is attracted to or held to a surface or member (e.g. adhesive; magnetic material). Others are possible. The basic function is to hold the seed for accurate and efficient tissue removal and isolate the seed from others, while maintaining identity of the seed.

In step 205, specific tissue is removed from a specified location of the seed. A number of methods can be used. It can be useful, in certain of the methods, to first orient the seed in a certain manner. In some examples, magnetic paint (FIG. 3, step 62) may assist in orientation of the seed to thereby further assist in removal of the specified tissue.

An example of tissue removal is with use of a laser. A laser can be precisely controlled in intensity. It also can be focused to a beam width that can be effectively used for removing only a relatively small area of tissue from one side of a seed, and to a relatively small, controlled depth. The laser beam can be operated in a variety of ways to effect tissue removal. An example is programmable raster scanning. The beam is controlled to move at a programmed speed and direction relative to the area to be removed. The laser beam can be focused upon and moved with precision across the seed to ablate the portion of the seed it strikes and remove tissue. Ablation may provide for removing or destroying, cutting, abrading, evaporating (vaporizing), clipping or otherwise. As used herein, ablation refers to such actions, or to analogous actions that remove or separate such seed tissue from the seed. In some instances, this results essentially in a candidate seed having some tissue removed to expose or allow access to internal tissue. The ablation may result in one piece or just a few pieces of removed tissue (more in the sense of cutting or chipping). Alternatively, the ablation may result in the removed tissue being essentially debris (more in the sense of fragments or very small particles, even dust-like, from abrasion, erosive processes, or the like). Alternatively, the ablation may result in the removed material evaporating, sublimating, or forming a plasma. A laser can function in these manners to remove specific tissue from the seed. As mentioned earlier, removed tissue can be collected for testing or analysis. Alternatively, testing or analysis of the remaining seed can be conducted as the tissue removal can be designed to expose or allow access to tissue in the remaining seed. In the case of corn, a laser beam can be controlled to remove an area of the pericarp to gain non-destructive access to underlying seed tissue(s), part(s), or structure(s) of interest, samples of which are collected and used for analysis. In addition a method such as a laser or mechanical slicing may be advantageous over a grinding method, as such methods may reduce potential for contamination between samples.

However, other methods of non-destructive seed tissue removal are possible. One example is a water jet or abrasive jet (e.g. commercially available from Berkeley Chemical Research, Inc., Berkeley, Calif. 94706-026; Flow International Corporation, Kent, Wash. USA; and others). Another is a grinding tool (e.g. Dremel brand MultiPro™ rotary tool) with appropriate sized bit and tip (e.g. engraving, cutting, grinding, carving, sanding, or routing bit tip available at a variety of commercial locations or on-line from Robert Bosch Tool Corporation). Of course, other methods of non-destructive seed tissue removal may be used, and different types may be better suited to different types of seeds, different environments, and other considerations.

In step 206, seed specific analysis is performed on an isolated seed. A number of analyses can be applied to the seed after tissue has been removed, or to the removed tissue from the seed. One example is genetic analysis. By methods known in the art, samples from the seed crown which contains pericarp and endosperm may be assayed for detection of nucleic acids from which genetic information about the seeds can be derived.

An example of one such method is as follows. The ablated seed can be immersed in a polymerase chain reaction (PCR) mixture in preparation for any number of PCR analyses. A detector can generate a signal representative of some aspect of the PCR from which genotyping can be derived. Details of such a signal and its use are well known. A variety of PCR detectors are commercially available. One example is an optical detector for PCR (e.g. Chromo4™ Real-Time PCR Detector from Bio-Rad Laboratories, Inc., Life Science Research Group, 2000 Alfred Nobel Drive, Hercules, Calif. 94547 USA). For example, a sliced portion of the endosperm ma be ground, extracted and amplified via PCR or other amplification process.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in, inter alia, Innis et al., eds. (1990) PCR Protocols: A Guide to Methods and Applications (Academic Press, New York); Innis and Gelfand, eds. (1995) PCR Strategies (Academic Press, new York); and Innis and Gelfand, eds. (1999) PCR Methods Manual (Academic Press, New York), herein incorporated by reference in their entirety. Known methods of PCR include, but are not limited to, methods using paired primers, nested primers, single specific primers, degenerate primers, gene-specific primers, vector-specific primers, partially-mismatched primers, and the like.

In hybridization techniques, all or part of the nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable marker. Methods for preparation of probes for hybridization and for construction of genomic libraries are generally known in the art.

Another analysis could be cellular level analysis. An example with respect to corn is described at Gabriella Consonni et al., “Genetic Analysis as a Tool to Investigate the Molecular Mechanisms Underlying Seed Development in Maize”, Annals of Botany 2005 96(3):353-362, which is incorporated by reference herein.

A still further example is nanoscale analysis. See, e.g., Georg H. H. et al., “Analysis of Detergent-Resistant Membranes in Arabidopsis. Evidence for Plasma Membrane Lipid”, Plant Physiol. January 2005; 137(1): 104-116, incorporated by reference herein.

Chemical analysis is another example. A variety of tests can be performed to identify a chemical trait of the tissue, or for other purposes. Other procedures or analyses are, of course, possible. The tissue removal step provides a sample for such analyses. One skilled in the art is familiar with the different analyses and testing that can be done on seed.

In step 207 the results of the analyses from step 206 are recorded. Typically, the results are recorded in an electronic database, but they may be recorded in other forms, such as ink on paper. In step 208 a determination is made as to whether the desired number of candidate seeds have been analyzed. If the desired number of candidate seeds have not yet been analyzed, an additional seed is selected is step 209 and steps 204-208 are repeated on the additional seed. Once the desired number of seeds have been analyzed, the results of the analyses are compared in step 210. Optionally, the analyses can be compared to results from previous assays for the same seed, and/or to data from parental or other non-related seed.

In step 211, a sample is selected based on the comparison of step 210. Once analysis has been completed, results or information from the analysis can be used to, for example, distinguish a seed from other seed, or identify a trait of the seed. This can be used to select one seed over another, or select a seed because of its trait. Of course, any number of other traits may be of interest. Examples of such traits may include herbicide tolerance, disease resistance, insect resistance, pest resistance, nutritional content traits, agronomic traits, industrial use traits, oil content traits, fatty acid content traits, environmental tolerance traits, or other traits of interest. By effective non-destructive sampling of a seed, and by an appropriate genotyping assay, seeds having particular traits (such as those traits indicated by the presence of genetic markers) can be identified.

As diagrammatically illustrated in FIG. 6, selection can be from a plurality of different candidate seed. The different candidate seed 1, 2 . . . , n are identified and collected (step 202). A first sample seed 1 (step 203) is processed through steps 204, 205, and 206, and a result of or data from the test of step 206 is recorded (step 207). One or more other sample seed (e.g. sample(s) 2, 3, . . . , n) are similarly processed (steps 204-206) and the test results stored for each (207) in correlation with their identifying information (202). This provides one basis for comparison between two or more of the samples (step 210) and subsequent selection between the two or more (step 211) of seed that is deemed desirable (e.g. for further research or commercial production). As indicated in FIG. 6, the comparison between samples can be based on any of variety of factors capable of analysis with seed specific tests of the samples, and/or optionally, parental or other non-related seed.

Importantly, non-destructive tissue removal and analysis allows such identification to be made without either planting the seed and waiting to test a tissue sample from its growing plant or having to use the land or greenhouse space, labor, and supplies to plant and grow the seed into plants. The controlled, precise, non-destructive removal of seed tissue for testing, or to gain access to relevant underlying tissue or structure for testing, allows analysis to make selections based on tissue of the seed, not on a plant grown from the seed. As can be appreciated, this represents a potential substantial savings in time, labor, and resources, including land resources, for selection processes for seed companies. The controlled, precise non-destructive tissue removal is capable of substantial automation, thus improving through put and efficiency of plant selection processes.

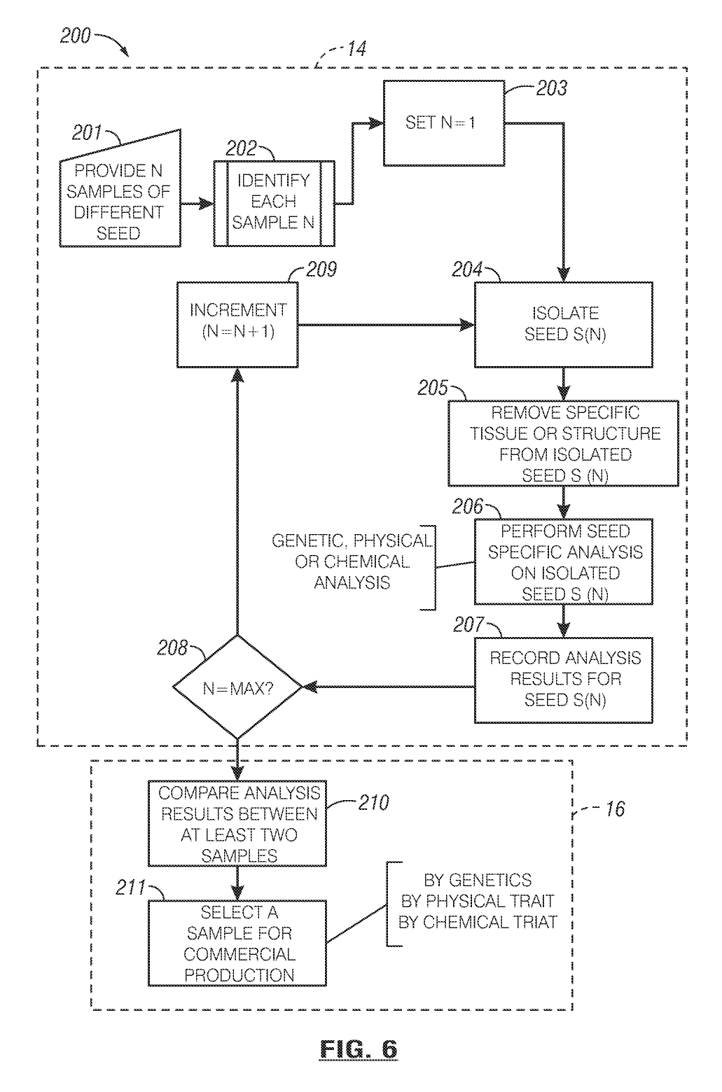

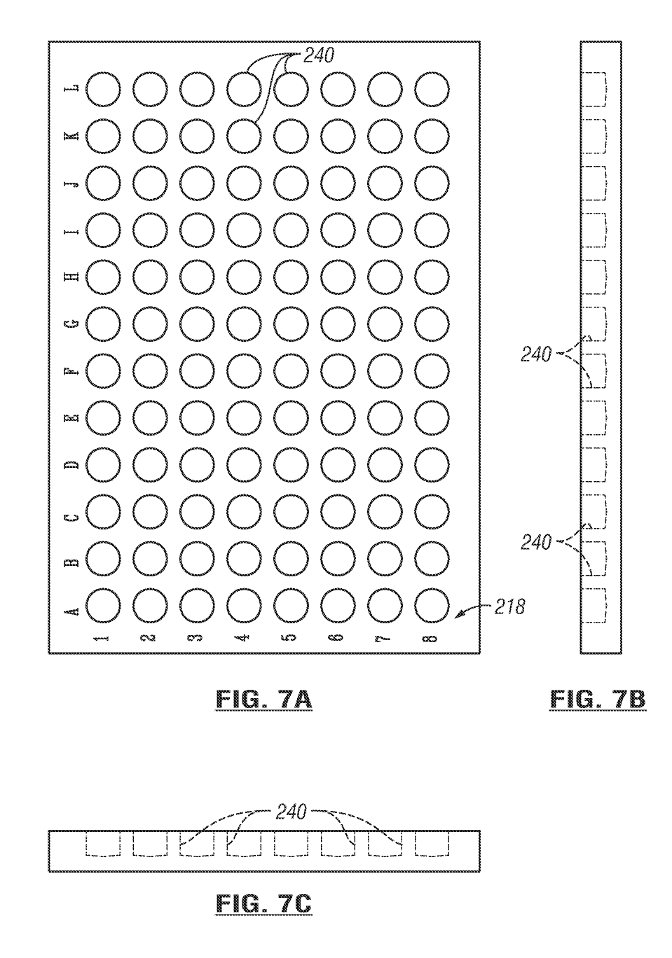

FIG. 7A-7C illustrate one manner in which seed may be stored. In FIG. 7A, seed is stored in individual wells 240 of a seed tray 218. The individual wells are indexed and indicia indicative of the indexing system may be located on the plate. Thus, for the seed tray 218 shown, seed may be referenced by column letter and row number. Of course, other indexing systems may be used and other configurations may be used, including circular or spiral configurations. In this manner, seed may be stored in an isolated and singulated manner so that each seed may be maintained. Seeds may also be stored in blister packs, see for example US2009/0077932, herein incorporated by reference. Of course various other types of containers of various shapes may be used including a bag, an envelope, a bubble tray, a reusable compartment tray, a breeder tray, a clamshell tray, seed tape, an assay tray, or any of the like.

Automated systems may be used for seed handling. An indexed storage system assists in facilitating automatic handling of the seed. The automated system should maintain seeds isolated and separated, such that each seed can be individually identified. The automated seed handling system may be implemented in any number of ways. This may include through vacuum systems, pressurized air systems, mechanical systems, or other types of systems. The re-arraying or re-positioning process (step 20 of FIG. 1), may involve removing seed from one storage container (such as a first seed tray) and placing the seed in a second storage container (such as a second seed tray). The second storage container may be a container adapted for use with a single seed planter which performs the planting operation.

Therefore, methods and systems for single seed management have been described. Numerous variations, options, and alternatives, are contemplated, such as may be apparent to one skilled in the art having the benefit of this disclosure.

What is claimed is:

1. A method for managing seed, comprising:
non-destructively sampling and testing tissue of individual seeds to obtain evaluation data for each of the individual seeds;
storing the evaluation data and seed identifiers associated with each of the individual seeds in a data store;
selecting a subset of individual seeds for planting at least partially based on the evaluation data; and
planting the subset of individual seeds.

2. The method of claim 1 wherein the evaluation data comprises at least one of genetic data, chemical data, parentage data, and physical data.

3. The method of claim 1 wherein the selecting the subset of individual seeds is at least partially based on at least one of genetic data, chemical data, physical data, and parentage data.

4. The method of claim 1 wherein the subset of individual seeds comprises seeds from a plurality of containers.

5. The method of claim 1 wherein the planting is based on a desired configuration of a plot or field.

6. The method of claim 5 wherein the desired configuration comprises relative placement of the subset of seeds.

7. The method of claim 5 wherein the desired configuration comprises geospatial position of each seed within the subset of individual seeds.

8. The method of claim 5 wherein the planting further includes modifying the desired configuration based on one or more exception conditions to provide an as-planted configuration.

9. The method of claim 1 wherein the planting being performed with a seed planter adapted to collect data about the planting of the individual seeds.

10. The method of claim 8 further comprising communicating the data about the planting of the individual seeds from the planter to the data store.

11. The method of claim 1 further comprising storing each of the subset of individual seeds in a first defined arrangement within one or more storage containers.

12. The method of claim 11 wherein a container identifier is provided for each of the one or more storage containers.

13. The method of claim 11 wherein at least one of the one or more storage containers provides for storing a plurality of singulated seeds.

14. The method of claim 13 wherein a position identifier is associated with each of the singulated seeds within each of the one or more storage containers.

15. The method of claim 11 further comprising moving the subset of individual seeds in the first defined arrangement within a first set of one or more storage containers storage container to a second set of one or more storage containers.

16. The method of claim 1 wherein the evaluation data includes data indicative of presence or absence of genetic markers associated with at least one trait selected from the group consisting of a yield trait, a disease resistance trait, an insect resistance trait, a herbicide resistance trait, and a tolerance of environmental stress trait.

17. The method of claim 1 wherein the non-destructively sampling includes orienting the individual seeds relative to a laser and ablating a portion of each of the individual seeds using the laser.

18. The method of claim 17 wherein magnetic paint on the individual seeds assists in orienting the individual seeds.

19. A method for management of seed data, comprising:
maintaining within a data store, data comprising at least one of: evaluation data for individual seeds, selection data for the individual seeds, storage data for the individual seeds, and planting data for the individual seeds;
sending a subset of the data from the data store for use in a work operation; and
performing the work operation on the individual seeds.

20. The method of claim 19 wherein the work operation is a data-driven process.

21. The method of claim 20 wherein the data-driven process comprises one of: a non-destructive tissue sampling process, a tissue evaluation process, a seed evaluation process, a seed re-positioning process, a pollination process, a field note-taking process, a plant tissue sampling process, and a planting process.

22. The method of claim 19 further comprising sending data associated with the work operation to the data store.

23. The method of claim 19 wherein the work operation is a tissue sampling operation.

24. The method of claim 19 wherein the work operation is a re-positioning operation for re-positioning one or more seeds from a first set of one or more storage containers container to a second set of one or more storage containers.

25. The method of claim 19 wherein at least one of the first container and the second container is from a set consisting of a bag, an envelope, a bubble tray, a reusable compartment tray, a breeder tray, a clamshell tray, seed tape, and an assay tray.

26. The method of claim 19 wherein the work operation is a seed planting operation.

27. The method of claim 25 wherein at least one of the first container and the second container is a blister pack.

28. The method of claim 19 wherein the sending comprises sending the subset of the data from the data store to an article of equipment adapted for performing the work operation.

29. The method of claim 19 further comprising receiving data about the work operation from a machine performing the work operation and storing the data about the work operation in the data store.

30. A system for management of seed on an individual seed basis, the system, comprising:
an evaluation subsystem for evaluating individual seeds that have had tissue removed and tested in a manner that is not destructive to the seeds to provide evaluation data for the seeds;
a selection subsystem for selecting a subset of the individual seeds at least partially based on the evaluation data;
a planting subsystem for planting the subset of individual seeds; and
a data store for storing seed management data, the seed management data including the evaluation data.

31. The system of claim 30 further comprising a data path between the data store and at least one of the subsystems.

32. The system of claim 30 further comprising a software application for managing at least one of priority for planting, location for planting, proximity for pollination, and field logistics.

33. The system of claim 30 further comprising a software application for laying out a planting configuration for the subset of the individual seeds.

34. The system of claim 30 further comprising a re-positioning subsystem for moving individual seeds between storage containers.

35. The system of claim 30 further comprising a container management software application for managing containers in which seed is stored.

36. A method for management of seed, comprising:
maintaining within a data store, data comprising: evaluation data for individual seeds, selection data for the individual seeds, storage data for the individual seeds, and planting data for the individual seeds;
providing an output to a user based on the data to assist the user in the management of the seed.

37. The method of claim 36 wherein the output is based on analysis of the data.

38. The method of claim 36 wherein the output is associated with at least one of seed quality control, regulatory reporting, sales and marketing, seed or plant characterization, and inventory management.

39. The method of claim 36, wherein the evaluation data is obtained by non-destructively sampling tissue from individual seeds and testing the sampled tissue.
